SEQUENCE LISTING (9 sequences)

SEQ ID NO: 1 - DNA, 1164 bp, Bacillus licheniformis
  CDS (1)..(1161): YugJ - Putative alcohol dehydrogenase
atggataactttacatattggaatcccacaaagctgatattcggccggggagaagttgaaaagctggcagaagaggtgaaacaatacggccgcaatgtcctgctcgtatacggcggaggcagcattaagcgaaacggtttatacgatcaagtcatttcaatccttgaaaaggcgggcgcgaccgtccatgaactgcccggcgtcgaaccgaatccgcgtgttgccactgtgaataaaggagttgcgatctgcaaagagaacgatattgactttcttttggcagtcggcggcggaagcgtcattgattgtacgaaagcaattgctgccggagcgaaatacgacggcgatgcgtgggatattgtgacgaaaaaacatattccggctgatgcgctgccgtttggaacagttttaacgttagcagcaacaggctctgaaatgaactcgggatctgtgatcacaaattgggaaaccaatgaaaaatacggctggggaagcccgctcgtattccctaaattttcaattcttgatccggtcaacacgtttaccgtcccgaaagaccacacgatttacggcattgtcgacatgatgtcccacgtgtttgagcaatattttcaccataccgaaaataccccttatcaggaccggatgtgcgaatccctgcttaaaacggtaattgaaacagctcctaagctcattgaagacctagaaaactatgagctgcgtgaaacgattctgtatacaggcaccattgcgctgaacggcatgctatcaatgggcgcacgcggagactgggcaacgcacaatatcgagcacgctgtttcagccgtatacgatattccgcatgcgggagggcttgcgattctgttcccgaattggatgaagcacacgctttccgagaacgtcggccgctttaaacagcttgccgtccgtgtttttgacgtagatgaaacaggaaaaaccgatcgcgaagtggcgcttgttggaatcgagaaactgtctgaattctggaccagccttggcgcgccaaatcgtcttgccgattatgacattacagatgagaagcttgatctcattgccgacaaagcgatggcaaacggcgaattcggccgctttaaaacgctgaataaagacgatgttctgtctattttgaaggcttctttataa

SEQ ID NO: 2 - protein, 387 aa, Bacillus licheniformis
MDNFTYWNPTKLIFGRGEVEKLAEEVKQYGRNVLLVYGGGSIKRNGLYDQVISILEKAGATVHELPGVEPNPRVATVNKGVAICKENDIDFLLAVGGGSVIDCTKAIAAGAKYDGDAWDIVTKKHIPADALPFGTVLTLAATGSEMNSGSVITNWETNEKYGWGSPLVFPKFSILDPVNTFTVPKDHTIYGIVDMMSHVFEQYFHHTENTPYQDRMCESLLKTVIETAPKLIEDLENYELRETILYTGTIALNGMLSMGARGDWATHNIEHAVSAVYDIPHAGGLAILFPNWMKHTLSENVGRFKQLAVRVFDVDETGKTDREVALVGIEKLSEFWTSLGAPNRLADYDITDEKLDLIADKAMANGEFGRFKTLNKDDVLSILKASL

SEQ ID NO: 3 - DNA, 4350 bp, Artificial sequence
  Plasmid pAN212b
aattcagatccttattgttcccgcgggacgtcgattcacaaaaataggcacacgaaaaacaagtaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcg

SEQ ID NO: 4 - DNA, 35 bp, Artificial sequence
  Primer yugJ1F
ataaaagtccgcggttgatcagacctgcgattccg

SEQ ID NO: 5 - DNA, 40 bp, Artificial sequence
  Primer yugJ2R
cagcgttttaaagcggccgatcgcttaatgctgcctccgc

SEQ ID NO: 6 - DNA, 40 bp, Artificial sequence
  Primer yugJ3F
gcggaggcagcattaagcgatcggccgctttaaaacgctg

SEQ ID NO: 7 - DNA, 35 bp, Artificial sequence
  Primer yugJ4R
tgcccggacgtcttttttcgtgaatggtatggtgg

SEQ ID NO: 8 - DNA, 930 bp, Artificial sequence
  PCR fragment yugJSOEpcr
ataaaagtccgcggttgatcagacctgcgattccgacaagcgcgtaaacggtccgtgctaaagcagacccttggccgccaaaaatggctgcaaccaagtcaaattgaaaaaatcctatcagtccccaattgattgctccgatgatcgttaaaaccaatgcaatacgttgaagtgcattcatttgttattcctcctattttgaaatcatatatttcacatttatagattgcgaccgatttggaaacattatacgcctgaggagcagatcgctttacagagcgttcggcgatttcccataatagtaaataaaggaggagatgtagatggataactttacatattggaatcccacaaagctgatattcggccggggagaagttgaaaagctggcagaagaggtgaaacaatacggccgcaatgtcctgctcgtatacggcggaggcagcattaagcgatcggccgctttaaaacgctgaataaagacgatgttctgtctattttgaaggcttctttataagatttcttgacggctcaaggagaaccgccattccttgagccgttgtttattgttatgtttttgacaaaaatgcaagtggaggctgaaaaaacatgcatttttggcgggcatcctccatcctcctttttttcgtcacttgatttaacgaaggagttcatataaagtgaaaagggaagaggctgtcgccgaaaatcgacatgttttaaaaccctttggcccgcactctcatgcaaatgaaagcgctggcgggtatttgaaagaaaacagctagtactggaggtttaataatatgacgcatgtccgttttgactactcaagagcattgccattctttaaagaacaggagcttacatatctgcgtgacttcgttaaagtcgcccaccataccattcacgaaaaaagacgtccgggca

SEQ ID NO: 9 - DNA, 5254 bp, Artificial sequence
  Plasmid pAN212b-yugJ
aattcagatccttattgttcccgcggttgatcagacctgcgattccgacaagcgcgtaaacggtccgtgctaaagcagacccttggccgccaaaaatggctgcaaccaagtcaaattgaaaaaatcctatcagtccccaattgattgctccgatgatcgttaaaaccaatgcaatacgttgaagtgcattcatttgttattcctcctattttgaaatcatatatttcacatttatagattgcgaccgatttggaaacattatacgcctgaggagcagatcgctttacagagcgttcggcgatttcccataatagtaaataaaggaggagatgtagatggataactttacatattggaatcccacaaagctgatattcggccggggagaagttgaaaagctggcagaagaggtgaaacaatacggccgcaatgtcctgctcgtatacggcggaggcagcattaagcgatcggccgctttaaaacgctgaataaagacgatgttctgtctattttgaaggcttctttataagatttcttgacggctcaaggagaaccgccattccttgagccgttgtttattgttatgtttttgacaaaaatgcaagtggaggctgaaaaaacatgcatttttggcgggcatcctccatcctcctttttttcgtcacttgatttaacgaaggagttcatataaagtgaaaagggaagaggctgtcgccgaaaatcgacatgttttaaaaccctttggcccgcactctcatgcaaatgaaagcgctggcgggtatttgaaagaaaacagctagtactggaggtttaataatatgacgcatgtccgttttgactactcaagagcattgccattctttaaagaacaggagcttacatatctgcgtgacttcgttaaagtcgcccaccataccattcacgaaaaaagacgtcgattcacaaaaataggcacacgaaaaacaagtaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcg

(19) United States
(12) Patent Application Publication
(10) Pub. No.: US 2010/0173286 A1
(43) Pub. Date: Jul. 8, 2010

(54) Mutant Cells Suitable for Recombinant Polypeptide Production

(75) Inventors: Jon Martin Persson, Bjaerred (SE); Allan Kent Nielsen, Soborg (DK); Niels Banke, Soborg (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsværd (DK)

(21) Appl. No.: 11/993,525

(22) PCT Filed: Jun. 20, 2006
(86) PCT No.: PCT/DK2006/000359

§ 371 Date: Dec. 21, 2007

Related U.S. Application Data
(60) Provisional application No. 60/694,540, filed on Jun. 28, 2005.

(30) Foreign Application Priority Data
Jun. 24, 2005 (DK) PA 2005 00936

(51) Int. Cl. C12Q 1/68; C12N 1/21; C12P 21/00

(52) U.S. Cl. 435/6; 435/252.3; 435/252.31; 435/69.1

(57) ABSTRACT

A mutated bacterial cell producing at least one heterologous polypeptide of interest, wherein said cell has a reduced expression-level of YugJ (SEQ ID NO: 2) or a homologue thereof when compared with an otherwise isogenic but non-mutated cell; methods for producing said cell, and methods for producing a polypeptide of interest using said cell.

### SEQUENCE LISTING

The present invention comprises a sequence listing.

### FIELD OF THE INVENTION

The invention relates to a mutated bacterial cell producing at least one heterologous polypeptide of interest, wherein said cell has a reduced expression-level of YugJ (SEQ ID NO: 2) or a homologue thereof when compared with an otherwise isogenic but non-mutated cell; methods for producing said cell, and methods for producing a polypeptide of interest using said cell.

### BACKGROUND OF THE INVENTION

Formation of polypeptide crystals/amorphous precipitate during fermentation is today seen frequently because the fermentation yields are getting higher and higher due to optimization of the fermentation recipes and/or due to identification/development or construction of more efficient production organisms.

In such cases, the polypeptides are fermented in yields that are above their solubility limit, meaning that they may be present in the culture broth in a partly precipitated form. The precipitate may be in the form of crystals or as amorphous precipitates.

This causes problems in recovery where special measures have to be taken to solubilize the crystals/amorphous precipitate before removing the cells and other solids from the culture broth. These measures often result in yield losses.

WO 2004/003187 discloses a method for fermenting a microorganism to produce a polypeptide of interest, wherein small amounts, e.g., 5% w/w, of one or more compounds selected from the group consisting of 1,2-propandiol, (monopropylene glycol; MPG), 1,3-propandiol, ethylene glycol, trehalose, xylitol, arabitol, dulcitol, mannitol, erythritol, cellobiose, sorbitol and a polyether having an average molecular weight less than 1000, are present during the fermentation, whereby the formation of crystals or amorphous precipitate of the polypeptide of interest can be avoided, significantly delayed or significantly reduced. By avoiding formation of polypeptide crystals/amorphous precipitate during fermentation, a much more simple recovery process can be used resulting in higher yields.

The MPG is only a very poor carbon source for most microorganisms or is very poorly metabolized by most microorganisms, or not metabolized at all, so it can be added before starting the fermentation and/or added during the fermentation without affecting the cell growth and productivity of the peptide of interest significantly.

However, some microorganisms degrade these added compounds, such as MPG, to a certain extent, and those microorganisms have to be supplied with a larger amount of the compounds to achieve the optimal effect. Since these compounds are somewhat costly, it is of interest to minimize the amounts needed to achieve the desired effect.

### SUMMARY OF THE INVENTION

Inactivation of the putative yugJ open reading frame in a Bacillus lichenifonnis enzyme production strain surprisingly lead to decreased degradation of monopropylene glycol (MPG) during fermentation. MPG is added to the growth medium to avoid or significantly reduce formation of enzyme crystals. Accordingly, less MPG needed to be added to the fermentation medium of the yugJ mutant strain to achieve the desired effect, thus production costs were reduced.

Based on sequence homology, the putative yugJ ORF was predicted to encode an alcohol dehydrogenase, most likely a butanol dehydrogenase. Numerous microorganisms in the literature have been found to comprise a yugJ homologue encoding alcohol or butanol dehydrogenases with amino acid sequences very similar to the predicted YugJ of the present invention, including, Bacillus subtilis, Bacillus cereus, Bacillus thuringiensis, Geobacillus kaustophilus, Bacillus clausii, Oceanobacillus iheyensis, Bacillus halodurans, and more.

Accordingly, in a first aspect the invention relates to a mutated bacterial cell producing at least one heterologous polypeptide of interest, wherein said cell has a reduced expression-level of YugJ (SEQ ID NO: 2) or a homologue thereof when compared with an otherwise isogenic but non-mutated cell,

Another aspect of the invention relates to a mutated bacterial cell producing at least one heterologous polypeptide of interest, wherein said cell has a reduced expression-level of an alcohol dehydrogenase comprising a polypeptide with an amino acid sequence at least 60% identical to SEQ ID NO: 2, or preferably at least 65%, 70%, 75%, 80%, 85%, 90%, 92%, 94%, 96%, 98%, or 99% identical to SEQ ID NO: 2, when compared with an otherwise isogenic but non-mutated cell.

Yet another aspect of the invention relates to a method for constructing a mutated bacterial cell, said method comprising the steps of:
a) mutating a bacterial cell; and
b) selecting a mutated cell which has a reduced expression-level of YugJ (SEQ ID NO: 2) or a homologue thereof when compared with an otherwise isogenic but non-mutated cell.

Still another aspect of the invention relates to a method for producing a polypeptide of interest, said method comprising the steps of:
a) cultivating a mutated bacterial cell producing at least one heterologous polypeptide of interest in a culture medium of at least 50 litres which comprises one or more compounds selected from the group consisting of 1,2-propandiol, 1,3-propandiol, ethylene glycol, trehalose, xylitol, arabitol, dulcitol, mannitol, erythritol, cellobiose, sorbitol and a polyether having an average molecular weight less than 1000, to the culture medium before and/or during fermentation, wherein said mutated cell has a reduced expression-level of YugJ (SEQ ID NO: 2) or a homologue thereof, and
b) isolating the polypeptide of interest.

A preferred embodiment of the invention relates to the mutant cell of any of the previous aspects, wherein the mutant cell shows a decreased ability to degrade one or more polyol, preferably selected from the group consisting of 1,2-propandiol (monopropylene glycol; MPG), 1,3-propandiol, ethylene glycol, trehalose, xylitol, arabitol, dulcitol, mannitol, erythritol, cellobiose, sorbitol and a polyether having an average molecular weight less than 1000, when compared with the otherwise isogenic but non-mutated cell.

### BRIEF DESCRIPTION OF DRAWINGS

FIG. 1 shows a schematic of plasmid pAN212b, a derivative of plasmid pSJ2739 (described in WO 99/41358), which is again derived from plasmid pE194, a naturally temperature-sensitive plasmid for replication. Plasmid pAN212b comprises the pE194 replicon, and a fragment derived from plasmid pUB110.

FIG. 2 shows a schematic of plasmid pAN212b-yugJ which consists of the yugJSOEpcr fragment cloned in the SacII-BsaHl sites of the temperature sensitive plasmid pAN212b which is shown in FIG. 1, the construction is described in the examples below.

### DETAILED DESCRIPTION OF THE INVENTION

### Microorganisms

The microorganism (microbial strain or cell) according to the invention may be obtained from microorganisms of any genus, such as those bacterial sources listed below. In a preferred embodiment the cell of the first aspects of the invention is a prokaryotic cell, preferably a Gram-positive cell, more preferably a Bacillus cell, and most preferably a Bacillus alkalophilus, Bacillus amyloliquefaciens, Bacillus brevis, Bacillus circulans, Bacillus clausii, Bacillus coagulans, Bacillus lautus, Bacillus lentus, Bacillus lichenifonnis, Bacillus megaterium, Bacillus stearothermophilus, Bacillus subtilis, or Bacillus thuringiensis cell.

### The Mutated Cell

In a preferred embodiment of the invention, the YugJ homologue comprises an amino acid sequence at least 60% identical to the sequence shown in SEQ ID NO: 2, preferably at least 65%, 70%, 75%, 80%, 85%, 90%, 92%, 94%, 96%, 98%, or 99% identical to SEQ ID NO: 2.

In another preferred embodiment the mutated cell of the invention is mutated in yugJ (SEQ ID NO: 1) or a homologue thereof; preferably the yugJ, and/or yugJ homologue encodes a polypeptide comprising an amino acid sequence at least 60% identical to the sequence shown in SEQ ID NO: 2, preferably at least 65%, 70%, 75%, 80%, 85%, 90%, 92%, 94%, 96%, 98%, or 99% identical to SEQ ID NO: 2; more preferably the yugJ homologue comprises a polynucleotide having a nucleotide sequence at least 60% identical to the sequence shown in SEQ ID NO: 1, preferably at least 65%, 70%, 75%, 80%, 85%, 90%, 92%, 94%, 96%, 98%, or 99% identical to SEQ ID NO: 1.

Preferably, the cell of the invention is mutated in at least one polynucleotide, where a subsequence having a size of at least 100 by of the at least one polynucleotide hybridizes with a polynucleotide having the sequence shown in SEQ ID NO: 1, or the respective complementary sequence, under medium stringency hybridization conditions.

In a preferred embodiment of the cell of the invention, yugJ or a homologue thereof, is partially or fully deleted from the chromosome; or yugJ or a homologue thereof, comprises at least one frameshift mutation or non-sense mutation.

A preferred result of these mutations is, that the cell of the invention has at least a two-fold reduced expression-level of YugJ or a homologue thereof, when compared with the otherwise isogenic but non-mutated cell; or that the cell has no measureable expression of YugJ or a homologue thereof, when compared with the otherwise isogenic but non-mutated cell.

### Polypeptide of Interest

In a preferred embodiment, the polypeptide of interest may be obtained from a bacterial or a fungal source.

For example, the polypeptide of interest may be obtained from a Gram positive bacterium such as a Bacillus strain, e.g., Bacillus alkalophilus, Bacillus amyloliquefaciens, Bacillus brevis, Bacillus circulans, Bacillus coagulans, Bacillus lautus, Bacillus lentus, Bacillus lichenifonnis, Bacillus megaterium, Bacillus stearothermophilus, Bacillus subtilis, or Bacillus thuringiensis; or a Streptomyces strain, e.g., Streptomyces lividans or Streptomyces murinus; or from a Gram negative bacterium, e.g., E. coli or Pseudomonas sp.

The polypeptide of interest may be obtained from a fungal source, e.g. from a yeast strain such as a Candida, Kluyveromyces, Pichia, Saccharomyces, Schizosaccharomyces, or Yarrowia strain, e.g., Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis strain.

The polypeptide of interest may be obtained from a filamentous fungal strain such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma strain, in particular the polypeptide of interest may be obtained from an Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride strain.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

For purposes of the present invention, the term “obtained from” as used herein in connection with a given source shall mean that the polypeptide of interest is produced by the source or by a cell in which a gene from the source has been inserted.

The polypeptide of interest may be a peptide or a protein. A preferred peptide according to this invention contains from 2 to 100 amino acids; preferably from 10 to 80 amino acids; more preferably from 15 to 60 amino acids; even more preferably from 15 to 40 amino acids.

In a preferred embodiment, the protein is an enzyme, in particular a hydrolase (class EC 3 according to Enzyme Nomenclature; Recommendations of the Nomenclature Committee of the International Union of Biochemistry). In a particular preferred embodiment the following hydrolases are preferred:

### Proteases

Suitable proteases include those of animal, vegetable or microbial origin. Microbial origin is preferred. Chemically modified or protein engineered mutants are included. The protease may be an acid protease, a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235 and 274.

Preferred commercially available protease enzymes include ALCALASE™, SAVINASE™, PRIMASE™, DURALASE™, ESPERASE™, RELASE™ and KANNASE™(Novozymes NS), MAXATASE™, MAXACAL™, MAXAPEM™, PROPERASE™, PURAFECT™, PURAFECT OXP™, FN2™, and FN3™ (Genencor International Inc.).

### Lipases

Suitable lipases include those of bacterial or fungal origin. Chemically modified or protein engineered mutants are included. Examples of useful lipases include lipases from Humicola (synonym Thermomyces), e.g. from H. lanuginosa (T. lanuginosus) as described in EP 258 068 and EP 305 216 or from H. insolens as described in WO 96/13580, a Pseudomonas lipase, e.g. from P. alcaligenes or P. pseudoalcaligenes (EP 218 272), P. cepacia (EP 331 376), P. stutzeri (GB 1,372,034), P. fluorescens, Pseudomonas sp. Strain SD 705 (WO 95/06720 and WO 96/27002), P. wisconsinensis (WO 96/12012), a Bacillus lipase, e.g. from B. subtilis (Dartois et al. (1993), Biochemica et Biophysica Acta, 1131, 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include LIPOLASE™, LIPOLASE ULTRA™ and LIPEX™ (Novozymes A/S).

### Amylases

Suitable amylases (alpha and/or beta) include those of bacterial or fungal origin. Chemically modified or protein engineered mutants are included. Amylases include, for example, alpha-amylases obtained from Bacillus, e.g. a special strain of B. lichenifonnis, described in more detail in GB 1,296,839.

Examples of useful amylases are the variants described in WO 94/02597, WO 94/18314, WO 96/23873, WO 97/43424, and WO 01/66712, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are DURAMYL™, TERMAMYL™, FUNGAMYL™, NATALASE™, TERMAMYL LC™, TERMAMYL SC™, LIQUIZYME-X™ and BAN™ (Novozymes A/S), RAPIDASE™ and PURASTAR™ (from Genencor International Inc.).

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include CELLUZYME™, CAREZYME™, and CAREZYME CORE™ (Novozymes A/S), CLAZINASE™, and PURADAX HA™ (Genencor International Inc.), and KAC-500(B)™ (Kao Corporation).

### Oxidoreductases

Oxidoreductases that may be treated according to the invention include peroxidases, and oxidases such as laccases, and catalases.

Other preferred hydrolases are carbohydrolases including MANNAWAY™. Other preferred enzymes are transferases, lyases, isomerases, and ligases.

### Expression Constructs for the Polypeptide of Interest

In a preferred embodiment the cell of the invention comprises one or more chromosomally integrated copies of a polynucleotide encoding the at least one heterologous polypeptide.

It is preferred that the at least one heterologous polypeptide of the invention is encoded by a polynucleotide which is transcribed from at least one heterologous promoter; preferably the at least one promoter comprises an artificial promoter. Suitable promoter constructs are disclosed in WO 93/10249 which is incorporated herein in its entirety by reference.

In addition, the preferred artificial promoter comprises one or more mRNA-stabilizing sequence, preferably derived from the cryllla promoter. Suitable constructs are described in WO 99/43835 which is incorporated herein in its entirety by reference.

### Fermentations

The present invention may be useful for any fermentation in industrial scale, e.g. for any fermentation having culture media of at least 50 litres, preferably at least 100 litres, more preferably at least 500 litres, even more preferably at least 1000 litres, in particular at least 5000 litres.

The bacterial strain or cell may be fermented by any method known in the art. The fermentation medium may be a complex medium comprising complex nitrogen and/or carbon sources, such as soybean meal, soy protein, soy protein hydrolysate, cotton seed meal, corn steep liquor, yeast extract, casein, casein hydrolysate, potato protein, potato protein hydrolysate, molasses, and the like. The fermentation medium may be a chemically defined media, e.g. as defined in WO 98/37179.

The fermentation may be performed as a batch, a fed-batch, a repeated fed-batch or a continuous fermentation process.

In a fed-batch process, either none or part of the compounds comprising one or more of the structural and/or catalytic elements is added to the medium before the start of the fermentation and either all or the remaining part, respectively, of the compounds comprising one or more of the structural and/or catalytic elements is fed during the fermentation process. The compounds which are selected for feeding can be fed together or separate from each other to the fermentation process.

In a repeated fed-batch or a continuous fermentation process, the complete start medium is additionally fed during fermentation. The start medium can be fed together with or separate from the structural element feed(s). In a repeated fed-batch process, part of the fermentation broth comprising the biomass is removed at time intervals, whereas in a continuous process, the removal of part of the fermentation broth occurs continuously. The fermentation process is thereby replenished with a portion of fresh medium corresponding to the amount of withdrawn fermentation broth.

In a preferred embodiment of the invention, a fed-batch, a repeated fed-batch process or a continuous fermentation process is preferred.

### Polyols

A very useful subgroup of carbohydrates, polyols, may be added to the fermentation according to the invention. Any polyol may be used. However, a polyol selected from the group consisting of 1,2-propandiol (monopropylene glycol; MPG), 1,3-propandiol, glycerol, ethylene glycol, xylitol, arabitol, dulcitol, mannitol, erythritol, cellobiose and sorbitol, is preferred. In particular, a slowly metabolizable polyol is preferred.

It is to be noted that some polyols, e.g. glycerol, are rather easily metabolized by most cells, but the uptake of e.g. glycerol can be blocked, meaning that glycerol may be used according to the present invention.

In a particular embodiment of the invention the polyol is added to the culture medium either prior to inoculation or after inoculation at an amount of at least 0.1% (w/w); in particular at an amount of at least 0.5% (w/w). The polyol is added to the culture medium either prior to inoculation or after inoculation at an amount of up to 10% w/w; preferably at an amount of up to 8% w/w; more preferably at an amount of up to 6% w/w; more preferably at an amount of up to 5% w/w; more preferably at an amount of up to 4% w/w; more preferably at an amount of up to 3% w/w; more preferably at an amount of up to 2% w/w; even more preferably at an amount of up to 1% w/w.

In some cases it may be an advantage to use a mixture of two or more polyols, e.g. glycerol and monopropylene glycol, or a mixture of a slowly metabolizable polyol and a slowly metabolizable carbohydrate.

### Extent of Metabolization

The following test may be used to check whether a microorganism, producing a polypeptide of interest, is not, or only to a low extent, able to metabolize a given compound:

A suitable media for the growth of the microorganism of interest is chosen. The media is characterized by the following parameters:

a: The media contains glucose as the only carbohydrate source.

b. When glucose is removed the media should only be able to support growth of a significantly lower biomass (less than 50%).

The growth of the microorganism of interest is then compared in the following 3 media:

I: Normal media (with glucose as the only carbohydrate source)
II: Media I without glucose
III: Media I without glucose, but with the same C-mol of the compound to be tested.

The growth is then followed for a period of 8 hr in the 3 above mentioned media. Inoculation is done with a concentration of biomass that will secure that the normal media is outgrown in 75% of the time frame. The amount of biomass is measured as optical density (OD) at 650 nm. OD obtained in the different media is measured.

The compound to be tested is defined as low metabolizable, if:

(OD_{III}-OD_{II})/(OD_{I}-OD_{II})<25%; preferably
(OD_{III}-OD_{II})/(OD_{I}-OD_{II})<20%; more preferably
(OD_{II}OD_{II})/(OD_{I}-OD_{II})<15%; more preferably
(OD_{III}-OD_{II})/(OD_{I}-OD_{II})<10%; more preferably
(OD_{III}-OD_{II})/(OD_{I}-OD_{II})<5%; more preferably

### (OD_{III}-OD_{II})/(OD_{I}-OD_{II})=0%

In Example 2 the fermentations are tested according to this procedure.

### Recovery of the Polypeptide of Interest

A further aspect of the invention concerns the downstream processing of the fermentation broth. After the fermentation process is ended, the polypeptide of interest may be recovered from the fermentation broth, using standard technology developed for the polypeptide of interest. The relevant downstream processing technology to be applied depends on the nature of the polypeptide of interest.

A process for the recovery of a polypeptide of interest from a fermentation broth will typically (but is not limited to) involve some or all of the following steps:

1) pre-treatment of broth (e.g. flocculation)

2) removal of cells and other solid material from broth (primary separation)

3) filtration

4) concentration

5) filtration

6) stabilization and standardization.

Apart from the unit operations listed above, a number of other recovery procedures and steps may be applied, e.g., pH-adjustments, variation in temperature, crystallization, treatment of the solution comprising the polypeptide of interest with active carbon, and use of various adsorbents.

By using the method of the invention the yield of the polypeptide of interest is much higher in the recovery when the crystal formation is reduced or eliminated by adding of, e.g. MPG, during fermentation.

The invention is further illustrated in the following examples, which are not intended to be in any way limiting to the scope of the invention as claimed.

### EXAMPLES

### Example 1

### Deletion of the yugJ Gene in a Bacillus lichenifonnis Strain

Unless otherwise mentioned the DNA manipulations and transformations were performed using standard methods of molecular biology (Sambrook et al. (1989) Molecular cloning: A laboratory manual, Cold Spring Harbor lab., Cold Spring Harbor, N.Y.; Ausubel, F. M. et al. (eds.) “Current protocols in Molecular Biology”. John Wiley and Sons, 1995; Harwood, C. R., and Cutting, S. M. (eds.) “Molecular Biological Methods for Bacillus”. John Wiley and Sons, 1990). Enzymes for DNA manipulations were used according to the specifications of the suppliers (e.g. restriction endonucleases, ligases etc. are obtainable from New England Biolabs, Inc.). Competent cells were prepared and transformed as described by Yasbin, R. E., Wilson, G. A. and Young, F. E. (1975) Transformation and transfection in lysogenic strains of Bacillus subtilis: evidence for selective induction of prophage in competent cells. J. Bacteriol, 121:296-304.

### Strains

B. lichenifonnis SJ1707: disclosed in WO 93/10249.
B. lichenifonnis SJ1707b: SJ1707 expressing a recombinant variant alpha-amylase enzyme disclosed in WO 01/66712.
B. lichenifonnis AN232: SJ1707b (ΔyugJ); this study.
B. subtilis PP289-5: Donor strain for conjugative transfer of plasmids with an origin of transfer, oriT, derived from pUB110 (described in WO 96/23073).

### Plasmids

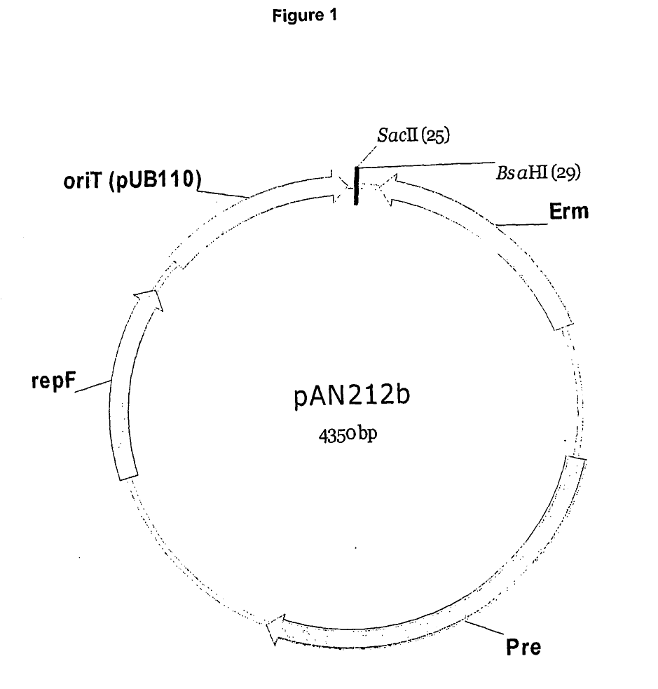

Plasmid pAN212b is a derivative of plasmid pSJ2739 (described in WO 99/41358), which is again derived from the well-known plasmid pE194, a naturally temperature-sensitive plasmid for replication. Plasmid pAN212b comprises the pE194 replicon, and a fragment derived from plasmid pUB110, as indicated in FIG. 1. The entire nucleotide sequence of pAN212b is shown in SEQ ID NO. 3.

### Primers

yugJ1F (SEQ ID NO. 4):
ataaaagtccgcggttgatcagacctgcgattccg
yugJ2R (SEQ ID NO. 5):
cagcgttttaaagcggccgatcgcttaatgctgcctccgc
yugJ3F (SEQ ID NO. 6):
gcggaggcagcattaagcgatcggccgctttaaaacgctg
yugJ4R (SEQ ID NO. 7):
tgcccggacgtcttttttcgtgaatggtatggtgg

Deletion of the yugJ gene in a Bacillus lichenifonnis strain may be performed based on the nucleotide sequence (SEQ ID NO. 1) by any of the standard methods well known in the art, e.g., as follows:

A PCR product is generated by use of the technique of splicing by overlap extension. PCR1 containing a yugJ upstream sequence, is generated by use of primers yugJ1 F and yugJ2R, in a PCR reaction with SJ1707 chromosomal DNA as template. PCR2, which contains a yugJ downstream sequence, is generated by use of primers yugJ3F and yugJ4R, in another PCR reaction with SJ1707 chromosomal DNA as template. The spliced product (930 bp, denoted yugJSOEpcr; shown in SEQ ID NO. 8), wherein the yugJ gene is reduced from 387aa to 51aa, is generated in a second-stage PCR using PCR1 and PCR2 as templates, and yugJ1F and yugJ4R as primers.

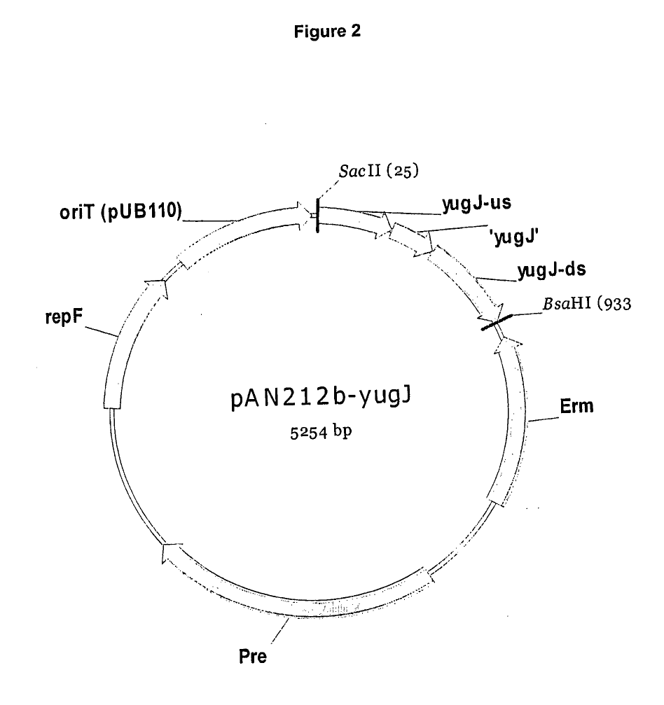

A plasmid denoted “deletion plasmid” is then constructed by cloning of yugJSOEpcr in the SacII-BsaHI sites of the temperature sensitive plasmid pAN212-resulting in the deletion plasmid pAN212b-yugJ, shown schematically in FIG. 2. The entire sequence of pAN212b-yugJ is shown in SEQ ID NO. 9.

The deletion plasmid is transformed into competent cells of the B. subtilis conjugation donor strain PP289-5 [ which contains a chromosomal dal-deletion, plasmid pBC16 (available from DSMZ ref. 4424; Kreft J, et al., 1978. Mol Gen Genet. Jun 1;162(11:59-67), and plasmid pLS20 (also available from DSMZ ref. 4449; Köhler, T. M., and Thorne, C. B. 1987. J. Bacteriol. 169: 5271-5278)] and conjugated to the B. lichenifonnis SJ1707b strain by use of standard methods (as described in WO 02/00907).

The yugJ deletion is then transferred from the deletion plasmid to the chromosome of the target B. lichenifonnis SJ1707b strain by double homologous recombination via PCR1 and PCR2, mediated by integration and excision of the temperature sensitive deletion plasmid (as described in WO 02/00907).

The yugJ-deleted strain is confirmed by generating a PCR fragment from chromosomal DNA with the primers yugJ1F and yugJ4R, werein the deletion is verified by standard nucleotide sequence analysis. The yugJ-deleted strain is denoted B. lichenifonnis AN232.

### Example 2

### Decreased MPG Degradation in a yugJ Deleted Strain

The two isogenic Bacillus lichenifonnis strains SJ1707b and AN232 (SJ1707b (ΔyugJ)) were fermented as follows:

### Media

In all cases unless otherwise described tap water was used. All media were sterilized by methods known in the art to ensure that the fermentations were run as mono-cultures.

First inoculum medium: LB agar was used as solid growth medium (as described in Ausubel, F. M. et al. (eds.) “Current protocols in Molecular Biology”. John Wiley and Sons, 1995). LB agar: 10 g/l peptone from casein; 5 g/l yeast extract; 10 g/l Sodium Chloride; 12 g/l Bacto-agar adjusted to pH 6.8 to 7.2. Premix from Merck was used.

Transfer buffer: M-9 buffer (deionized water is used): Di-Sodiumhydrogenphosphate, 2H2O 8.8 g/l; Potassiumdihydrogenphosphate 3 g/l; Sodium Chloride 4 g/l; Magnesium sulphate, 7H2O 0.2 g/l.

Inoculum shake flask medium (concentration is before inoculation): PRK-50: 110 g/l soy grits; Di-Sodiumhydrogenphosphate, 2H2O 5 g/l; pH adjusted to 8.0 with NaOH/H3PO4 before sterilization.

Make-up medium (concentration is before inoculation): Tryptone (Casein hydrolysate from Difco) 30 g/l; Magnesium sulphate, 7H2O 4 g/l; Di-Potassiumhydrogenphosphate 7 g/l; Di-Sodiumhydrogenphosphate, 2H2O 7 g/l; Di-Ammoniumsulphate 4 g/l; Citric acid 0.78 g/l; Vitamins (Thiamin-dichlorid 34.2 mg/l; Riboflavin 2.9 mg/l; Nicotinic acid 23 mg/l; Calcium D-pantothenate 28.5 mg/l; Pyridoxal-HCl 5.7 mg/l; D-biotin 1.1 mg/l; Folic acid 2.9 mg/l); Trace metals (MnSO4, H2O 39.2 mg/l; FeSO4, 7H2O 157 mg/l; CuSO4, 5H2O 15.6 mg/l; ZnCl2 15.6 mg/l); Antifoam (SB2121) 1.25 ml/l; pH adjusted to 6.0 with NaOH/H3PO4 before sterilization.

Feed medium: Glucose, 1H2O 820 g/l;

### Procedure

First the strains were grown on LB agar slants 1 day at 37° C. The agar was then washed with M-9 buffer, and the optical density (OD) at 650 nm of the resulting cell suspensions were measured.

The inoculum shake flasks (PRK-50) were inoculated with an inoculum of OD (650 nm)×ml cell suspension=0.1. The shake flasks were then incubated at 37° C. at 300 rpm for 20 hr.

The fermentation in the main fermentor (fermentation tank) was started by inoculating the main fermentor with the growing culture from a shake flask. The inoculated volume was 10% of the make-up medium (80 ml for 800 ml make-up media).

Standard lab fermentors were used equipped with a temperature control system, pH control with ammonia water and phosphoric acid, dissolved oxygen electrode to measure>20% oxygen saturation through the entire fermentation. Fermentation parameters were:

Temperature: 41° C.

The pH was kept between 6.8 and 7.2 using ammonia water and phosphoric acid

Control: 6.8 (ammonia water); 7.2 phosphoric acid

Aeration: 1.5 liter/min/kg broth weight

Agitation: 1500 rpm

Feed strategy:
0 hr. 0.05 g/min/kg initial broth after inoculation
8 hr. 0.156 g/min/kg initial broth after inoculation
End 0.156 g/min/kg initial broth after inoculation

### Results:

Two fermentations of SJ1707b (yugJ wildtype) and AN 232 (yugJ deletion mutant), respectively, were run in parallel. 2% MPG was added to both fermentations at 24 h and at 50 h 20 min. The results (shown in table 1) revealed a decreased MPG metabolism in the yugJ deleteted strain AN232 (in the time period 30 h-93 h) compared to the otherwise isogenic strain SJ1707b. Conclusively, by using a yugJ deleted strain the fermentation costs can be reduced, since less MPG needs to be added for the reduction of crystal formation.

TABLE 1
MPG concentration (%) in the fermentation broths of
fermentation A (SJ1707b) and B (AN232; yugJ mutant).
2% MPG was added at 24 h and at 50 h 20 min.

MPG concentration (%)

| Time | SJ1707b | AN232 |
|---|---|---|
| 24 h | 1.5 | 1.6 |
| 25 h 45 min | 1.1 | 1.1 |
| 29 h 30 min | 0.8 | 0.8 |
| 50 h | 0.5 | 0.7 |
| 68 h | 1.4 | 1.6 |
| 93 h | 1.0 | 1.5 |

1-57. (canceled)

58. A mutated bacterial cell producing at least one heterologous polypeptide of interest, wherein said cell has a reduced expression-level of YugJ (SEQ ID NO: 2), or a homologue thereof, when compared with an otherwise isogenic but non-mutated cell.

59. The cell according to claim 58, which is prokaryotic cell, preferably a Gram-positive cell.

60. The cell according to claim 58, which is a Bacillus cell.

61. The cell according to claim 58, which is a Bacillus alkalophilus, Bacillus amylohquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, or Bacillus thuringiensis cell.

62. The cell according to claim 58, wherein the YugJ homologue comprises an amino acid sequence at least 70% identical to the sequence shown in SEQ ID NO: 2.

63. The cell according to claim 58, which is mutated in yugJ (SEQ ID NO: 1) or a homologue thereof.

64. The cell according to claim 58, which is mutated in yugJ or a homologue thereof encoding a polypeptide comprising an amino acid sequence at least 90% identical to the sequence shown in SEQ ID NO: 2.

65. The cell according to claim 58, which is mutated in yugJ or a homologue thereof comprising a polynucleotide having nucleotide sequence at least 90% identical to the sequence shown in SEQ ID NO: 1.

66. The cell according to claim 58, which is mutated in at least one polynucleotide, where a subsequence having a size of at least 100 bp of the at least one polynucleotide hybridizes with a polynucleotide having the sequence shown in SEQ ID NO 1, or the respective complementary sequence, under medium stringency hybridization conditions.

67. The cell according to claim 58, which is mutated in yugJ or a homologue thereof, and in which yugJ or a homologue thereof is partially or fully deleted from the chromosome.

68. The cell according to claim 58, which is mutated in yugJ or a homologue thereof and in which yugJ or a homologue thereof comprises at least one frameshift mutation or non-sense mutation.

69. The cell according to claim 58, which has at least a two-fold reduced expression-level of YugJ or a homologue thereof, when compared with the otherwise isogenic but non-mutated cell.

70. The cell according to claim 58, which has no measureable expression of YugJ, or a homologue thereof, when compared with the otherwise isogenic but non-mutated cell.

71. The cell according to claim 58, wherein the at least one heterologous polypeptide comprises an enzyme.

72. The cell according to claim 58, wherein the at least one heterologous polypeptide comprises an enzyme selected from the group consisting of a lyase, a ligase, a hydrolase, an oxidoreductase, a transferase, and an isomerase.

73. The cell according to claim 58, which comprises one or more chromosomally integrated copies of a polynucleotide encoding the at least one heterologous polypeptide.

74. The cell according to claim 58, wherein the at least one heterologous polypeptide is encoded by a polynucleotide which is transcribed from at least one heterologous promoter.

75. The cell according to claim 58, wherein the at least one heterologous polypeptide is encoded by a polynucleotide which is transcribed from at least one heterologous promoter and wherein the at least one promoter comprises an artificial promoter.

76. A method for constructing a mutated bacterial cell, said method comprising the steps of:
a) mutating a bacterial cell; and
b) selecting a mutated cell which has a reduced expression-level of YugJ (SEQ ID NO: 2) or a homologue thereof when compared with an otherwise isogenic but non-mutated cell.

77. A method for producing a polypeptide of interest, said method comprising the steps of:
a) cultivating a mutated bacterial cell producing at least one heterologous polypeptide of interest in a culture medium of at least 50 litres which comprises one or more compounds selected from the group consisting of 1,2-propandiol, 1,3-propandiol, ethylene glycol, trehalose, xylitol, arabitol, dulcitol, mannitol, erythritol, cellobiose, sorbitol and a polyether having an average molecular weight less than 1000, to the culture medium before and/or during fermentation, wherein said mutated cell has a reduced expression-level of YugJ (SEQ ID NO: 2) or a homologue thereof, and
b) isolating the polypeptide of interest.
